(19) United States
(12) Patent Application Publication
(10) Pub. No.: US 2025/0333709 A1
(43) Pub. Date: Oct. 30, 2025

(54) BRAIN ENDOTHELIAL CELLS AND METHODS OF MAKING

(71) Applicant: President and Fellows of Harvard College, Cambridge, MA (US)

(72) Inventors: Jung Hyun Lee, Cambridge, MA (US); Lee L. Rubin, Cambridge, MA (US)

(21) Appl. No.: 18/870,315

(22) PCT Filed: May 30, 2023
(86) PCT No.: PCT/US2023/023906

§ 371 Date: Nov. 27, 2024

Related U.S. Application Data
(60) Provisional application No. 63/346,821, filed on May 27, 2022.

(51) Int. Cl. C12N 5/071

(52) U.S. Cl. CPC C12N 5/069

(57) ABSTRACT

Non-naturally occurring in vitro-derived peripheral endothelial cells and brain endothelial cells and methods of making and using the described cells are disclosed.

### RELATED APPLICATION

This application claims the benefit of and priority to U.S. Provisional patent application No. 63/346,821, filed May 27, 2022, the entire teachings of which are incorporated herein by reference.

### GOVERNMENT SUPPORT

This invention was made with government support under NS117407 and AG072086 awarded by the National Institutes of Health. The government has certain rights in the invention.

### BACKGROUND OF THE INVENTION

During development, endothelial cells (ECs) migrate into the brain, where, under the inductive influence of the brain environment, they differentiate into the cells that form the blood-brain barrier (BBB). Dysfunction of the BBB has been associated with cognitive impairment in aging and dementia.

To date the field has utilized two types of in vitro cell models: primary brain EC and iPSC-derived brain microvascular ECs (iBMEC), both of which have inherent limitations. Primary brain ECs are expensive, hard to obtain in large quantities, and lose some brain EC-specific characteristics after isolation. Moreover, previously described iBMECs are able to mimic some of brain EC characteristics, but the cell identity is much closer to epithelial cells than to endothelial cells.

Accordingly, to provide a physiologically relevant brain EC model to carry out detailed studies of the molecular and cellular changes that accompany BBB dysfunction and to produce cells that may be useful in cell therapy applications, more reliable methods are essential.

### SUMMARY OF THE INVENTION

As described herein, the development trajectory of ECs was recapitulated in vitro to differentiate human pluripotent cells (hPSCs) into brain ECs. First, hPSCs were differentiated into peripheral ECs by mesodermal commitment followed by expansion and isolation of CD144-positive cells. Next, by modulating Wnt, TGF-beta, and STAT3 signaling pathways, peripheral ECs were converted to brain EC-like cells. In addition, overexpression of brain EC transcription factors, along with epigenetic modulation, further augmented brain EC phenotypes including upregulation of GLUT1, MFSD2A, ABCB1, CLDN5, downregulation of PLVAP, and decreased permeability. This work provides an improved human brain EC model and tools that can be used for understanding the development and maintenance of BBB properties in health and disease and for providing cells useful in cell therapy applications. Work described herein can be used to analyze molecular functions, development of the BBB, transport of various substances across the BBB, and interaction of ECs with other brain cell types

Accordingly, in one embodiment the invention relates to a method of producing a cell population comprising peripheral endothelial cells in vitro comprising inducing one or more pluripotent cells to undergo mesodermal commitment, contacting the one or more cells that have undergone mesodermal commitment with VEGF, and purifying and optionally expanding CD144+ cells.

In another embodiment, the invention relates to a method of producing a cell population comprising brain endothelial cells in vitro comprising inducing one or more pluripotent cells to undergo mesodermal commitment, contacting the one or more cells that have undergone mesodermal commitment with VEGF, purifying and optionally expanding CD144+ cells, and modulating one or more signaling pathways selected from the group consisting of the Wnt signaling pathway, the TGF-beta signaling pathway, and the STAT3 signaling pathway in the purified and optionally expanded CD144+ cells.

In another embodiment, the invention relates to a method of producing a cell population comprising brain endothelial cells in vitro comprising contacting one or more cells that have undergone mesodermal commitment with VEGF, purifying and optionally expanding CD144+ cells, and modulating one or more signaling pathways selected from the group consisting of the Wnt signaling pathway, the TGF-beta signaling pathway, and the STAT3 signaling pathway in the purified and optionally expanded CD144+ cells.

In another embodiment, the invention relates to a method of producing a cell population comprising brain endothelial cells in vitro comprising modulating one or more signaling pathways selected from the group consisting of the Wnt signaling pathway, the TGF-beta signaling pathway, and the STAT3 signaling pathway in purified and optionally expanded CD144+ cells.

In some embodiments of the method of producing a cell population comprising brain endothelial cells, the method further comprises inhibiting HDAC expression in the purified and optionally expanded CD144+ cells. In some embodiments, inhibiting HDAC expression comprises contacting the purified and optionally expanded CD144+ cells with one or more agents selected from the group consisting of entinostat, panobinostat, and quisinostat.

In some embodiments of the method of producing a cell population comprising brain endothelial cells, the method further comprises increasing expression of one or more brain EC-specific transcription factors in the purified and optionally expanded CD144+ cells. In some embodiments, the one or more EC-specific transcription factors is selected from the group consisting of TCF7, PPARd, ZIC3, FOXC1, FOXL2, FOXF2, FOXQ1, and LEF1.

In some embodiments of the described methods above, the one or more pluripotent cells are human cells. In some embodiments, the one or more pluripotent cells are selected from the group consisting of embryonic stem cells and induced pluripotent stem cells. In certain embodiments the one or more pluripotent cells are induced pluripotent stem cells. In some embodiments the one or more pluripotent cells are cells that express NANOG.

In some embodiments of the invention, inducing one or more pluripotent cells to undergo mesodermal commitment comprises treating the one or more pluripotent cells with one or more agents in accordance with the mesoderm commitment pathways shown in FIG. 7. In some embodiments, inducing one or more pluripotent cells to undergo mesodermal commitment contacting the one or more pluripotent cells with Chir99021 on day 1. In some embodiments, inducing one or more pluripotent cells to undergo mesodermal commitment comprises contacting the one or more pluripotent cells with BMP4 on day 1. In certain embodiments, inducing one or more pluripotent cells to undergo mesodermal commitment comprises contacting the one or more pluripotent cells with Chir99021 and BMP4 on day 1. In some embodiments, mesodermal commitment comprises expression of PAX2 and BRACHYURY by the contacted cells. In certain embodiments, PAX2 expression peaks at about day 2. In certain embodiments, BRACHYURY expression peaks at about day 3. In some embodiments, contacting the one or more cells that have undergone mesodermal commitment with VEGF occurs on about day 4. In some embodiments, the cells begin expressing one or more of FLT1/VEGFR1 and KDR/VEGFR2 after being contacted with VEGF, and in certain embodiments expression of FLT1/VEGFR1 and KDR/VEGFR2 peaks on about day 5.

Some embodiments of the methods further comprise contacting the one or more cells that have undergone mesodermal commitment with FSK, e.g., on about day 4.

In some embodiments of the invention, the one or more cells begin to express one or more pan-EC markers on about day 5. For example, the one or more pan-EC markers can be selected from the group consisting of CDH5/VE-CAD, CD31/PECAM1, and SOX17, but the pan-EC markers are not limited to this group. In certain embodiments, the cells do not significantly express one or more ectodermal epithelial cell markers by day 4 or day 5. For example, the one or more ectodermal epithelial cell markers can be selected from the group consisting of PAX6, MAP2, CDH1 and EPCAM, but the ectodermal epithelial markers are not limited to this group.

In certain embodiments, purifying CD144+ cells comprises magnetic cell sorting using microbeads. In some embodiments the methods further comprise purifying CD31+ cells, e.g., by magnetic cell sorting using microbeads. In certain embodiments the method comprises purifying CD144+/CD31+ cells, and in these embodiments the method can comprise sequential cell sorting, e.g., using magnetic cell sorting with microbeads.

In some embodiments, the one or more signaling pathways is the Wnt signaling pathway. In some embodiments, the one or more signaling pathways is the TGF-beta signaling pathway. In some embodiments, the one or more signaling pathways is the STAT3 signaling pathway. In certain embodiments, two of the Wnt signaling pathway, the TGF beta signaling pathway, and the STAT3 signaling pathway are modulated. In some embodiments, the TGF beta signaling pathway, and the STAT3 signaling pathway are all modulated.

In some embodiments, modulation of the Wnt signaling pathway is achieved by contacting the purified and optionally expanded CD144+ cells with one or more agents selected from the group consisting of EGM2, WNT1, WNT3A, WNT5A, WNT5B and WNT7A. In some embodiments, modulation of the Wnt signaling pathway upregulates GLUT1. In some embodiments modulation of the Wnt signaling pathway downregulates PLVAP.

In certain embodiments, modulation of the one or more signaling pathways is achieved by contacting the purified and optionally expanded CD144+ cells with one or more agents selected from the group consisting of EC medium, ciliary neurotrophic factor (CNTF), CNTF receptor alpha (CNTFRa), pericyte medium (PM), pericyte conditioned medium (PCM), concentrated PM, and concentrated PCM. In some embodiments, modulation increases CLDN5 expression.

In certain embodiments, modulation of the TGF beta signaling pathway is achieved by contacting the purified and optionally expanded CD144+ cells with one or more TGFBR1 inhibitors. In some embodiments, modulation of the TGF beta signaling pathway is achieved by contacting the purified and optionally expanded CD144+ cells with one or more agents selected from the group consisting of RepSox, SB431542, SB525334 and Galunisertib. In some embodiments, modulation increases CLDN5 expression.

In certain aspects the invention relates to a non-naturally occurring in vitro-derived peripheral endothelial cell or a population of cells comprising a non-naturally occurring in vitro-derived peripheral endothelial cell. In other embodiments, the invention relates to a non-naturally occurring in vitro-derived brain endothelial cell or a population of cells comprising a non-naturally occurring in vitro-derived brain endothelial cell. In some embodiments the cells are isolated. In certain embodiments the non-naturally occurring in vitro-derived brain endothelial cell is characterized by one or more of EC marker expression, monolayer formation, increased expression of CLDN5 in comparison with a non-EC cell, increased expression of OCLN in comparison with a non-EC cell, increased expression of GLUT1 in comparison with a non-EC cell, increased expression of ABCB1 in comparison with a non-EC cell, increased expression of MFSD2A in comparison with a non-EC cell, and decreased expression of PLVAP in comparison with a non-EC cell. In certain aspects the invention relates to a cell line comprising a non-naturally occurring in vitro-derived peripheral endothelial cell or a cell line comprising a non-naturally occurring in vitro-derived brain endothelial cell. In some embodiments the cell is genetically modified (e.g., to secrete a particular protein or peptide or to protect the cell from immune challenge by a recipient); in other embodiments the cell is not genetically modified. In some embodiments the cell is encapsulated in a manner to protect it from immune attack upon administration to a patient.

The invention also relates to methods of using the cells or cell populations in methods of cell therapy, e.g., to form vessels or vessel-like structures in the brain such as for revascularization of injured brain or aging brain (e.g., to improve cognitive function) or for repair of the BBB. Cells and cell populations described herein can also be engineered to secrete agents and used for targeted delivery of such agents to the brain. The invention also relates to the use of the cells and cell populations described herein as tools to be used in molecular investigations and screening.

In some aspects, the disclosure provides a method for the treatment of a subject in need thereof, the method comprising administering to a subject a composition comprising a non-naturally occurring in vitro-derived peripheral endothelial cell or a population of cells comprising a non-naturally occurring in vitro-derived peripheral endothelial cell or a non-naturally occurring in vitro-derived brain endothelial cell or a population of cells comprising a non-naturally occurring in vitro-derived brain endothelial cell produced according a method described herein. In some embodiments, the peripheral endothelial cell or brain endothelial cells are produced from a population of pluripotent stem cells obtained from the same subject to whom the peripheral endothelial cell or brain endothelial cells are administered.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, NJ, 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, MD) and National Center for Biotechnology Information, National Library of Medicine (Bethesda, MD), as of May 1, 2010, World Wide Web URL: http://www.ncbi.nlm.nih.gov/omim/and in Online Mendelian Inheritance in Animals (OMIA), a database of genes, inherited disorders and traits in animal species (other than human and mouse), at http://omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1E show that iPSC-derived endothelial cells have the potential to develop into brain endothelial cells. FIG. 1A is a brief summary of peripheral endothelial cell differentiation from iPSC. Following mesoderm induction, endothelial cell lineage cells are expanded and isolated to obtain homogeneous population. Immunostainings of CD31 and VE-Cad show that differentiated cells can form a homogeneous monolayer. Scale bars, 100 um. FIG. 1B shows a time-course gene expression analysis during iPSC-EC differentiation. FIG. 1C shows images of transplanted GFP-labelled iPSC-EC in mouse brain, showing that these cells can form vascular structures in vivo. 28,000 cells were injected into the cortex region of neonatal mice. Brain sections were prepared 5 days after injection. Scale bars, 200 um. FIG. 1D demonstrates strategies for inducing brain EC differentiation from peripheral iPSC-ECs. The first approach is to apply extracellular signals that may induce brain EC signals or co-culture with other brain cells. The second approach is transcriptional reprogramming by overexpressing brain ECC-specific transcription factors. FIG. 1E shows the criteria for a successful brain EC model.

FIGS. 2A-2C demonstrate that Wnt signaling activation upregulates GLUT1 and downregulates PLVAP in iPSC-ECs. FIG. 2A shows immunostaining of GLUT1 upon treatment with different Wnt ligands (100 ng/ml for 2 days). FIG. 2B shows the mRNA levels of GLUT1 and PLVAP in WNT3A—or Chir99021—treated iPSC-ECs. FIG. 2C shows beta-catenin protein localization and expression of EC marker proteins after Wnt activator-treated iPSC-ECs. CTNNB1::GFP iPSC line (Allen Cell, AICS-0058-067iPSC) was used to visualize subcellular localization of beta-catenin protein. *, P<0.05 vs. EGM2; **, P<0.01 vs. EGM2. Scale bars, 100 um.

FIGS. 3A-3E demonstrate that CNTF-STAT3 pathway activation increases CLDN5 expression. FIG. 3A illustrates iPSC-ECs exposed to either fresh astrocyte medium (AM) or astrocyte-conditioned medium (ACM). Conditioned medium was collected for 48 hours. Both AM and ACM were treated as 1:1 mixture with EC medium (EGM2). FIG. 3B shows immunostaining of CLDN5 and PLVAP in CNTF-treated iPSC-ECs. Human recombinant ciliary neurotrophic factor (CNTF), 20 ng/ml; human recombinant CNTF receptor alpha (CNTFRa), 100 ng/ml. FIG. 3C shows Western blots of p-STAT3 during time course of CNTF and CNTFR treatment in iPSC-ECs. FIG. 3D shows immunostaining images showing the effect of pericyte medium (PM), pericyte-conditioned medium (PCM), concentrated PC, and concentrate PCM. PM and PCM were treated as 1:1 mixture with EGM2 for 2 days. FIG. 3E shows Western blots of p-STAT3 after concentrated PCM treatment. Scale bars, 100 um.

FIGS. 4A-4D demonstrate that inhibitors of TGF beta signaling increase CLDN5 expression in iPSC-ECs. Representative immunostaining images of iPSC-ECs after treatment with TGFBR1 inhibitor RepSox (10 uM, FIG. 4A) and human recombinant TGF beta (FIG. 4B). FIG. 4c shows immunostaining images of iPSC-ECs in the presence of different TGFBR1 inhibitors. All compounds were treated at 10 uM concentrations for 2 days. FIG. 4D shows iPSC-ECs exposed to a combination of WNT3A, CNTF, and RepSox. Each signaling factor has an additive effect on brain EC gene regulation. Scale bars, 100 um.

FIGS. 5A-5C show overexpression of brain EC-specific transcription factors and inhibition of HDAC induce a subset of brain EC-enriched genes. FIG. 5A shows gene expression analysis of brain EC genes in cells overexpressing brain EC-specific transcription factors. FIG. 5B shows the mRNA levels of brain EC genes upon HDAC inhibitor treatment in combination with WNT3A, CNTF, and RepSox (WCNR) in iPSC-ECs. FIG. 5C shows mRNA expression levels of brain EC genes in the presence and the absence of HDAC inhibitor quisinostat (10 nM), showing that Wnt pathway activator and PKC activator can induce expression of MFSD2A and ABCB1 in iPSC-ECs. Chir99021, 5 uM; WNT3A, 100 ng/ml; IndoV, 100 nM; PMA, 100 nM, Q, quisinostate 10 nM; 48 hours. *, P<0.05 vs. Control; **, P<0.01 vs. Control.

FIG. 6 shows that transplanted iPSC-ECs form vessel-like structures in mouse brain (7 dpi, P9 NSG mouse). The images are from whole mount brain imaging of tdTomato-labeled iPSC-ECs after PBS perfusion.

FIG. 7 illustrates the multiple pathways to mesodermal commitment from wikipathways.org. The model depicts mesodermal specification based on the literature and highly enriched gene expression profiles via comparison across dozens of independent induced and embryonic pluripotent stem cell lines, following differentiation to multiple lineages (ectoderm, mesoderm, endoderm, embryoid body). The underlying genomic data can be obtained from: https://www.synapse.org/#!Synapse:syn1773109. Proteins on this pathway have targeted assays available via the [https://assays.cancer.gov/available_assays?wp_id=WP2857 CPTAC Assay Portal].

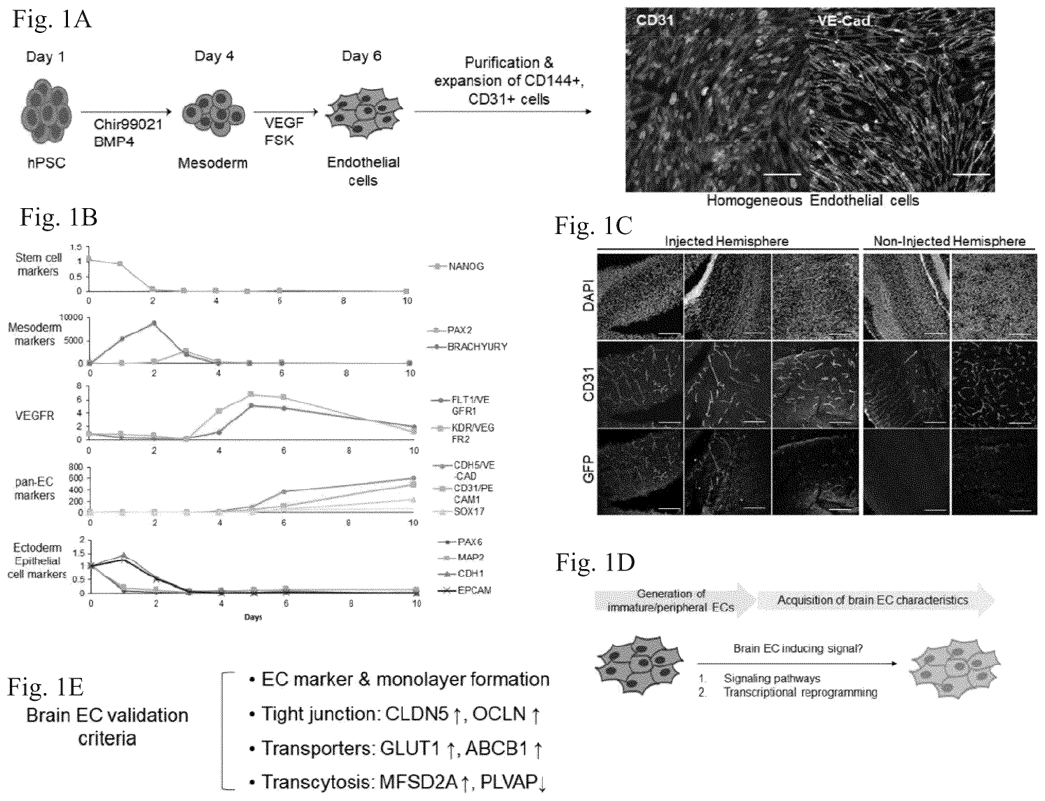

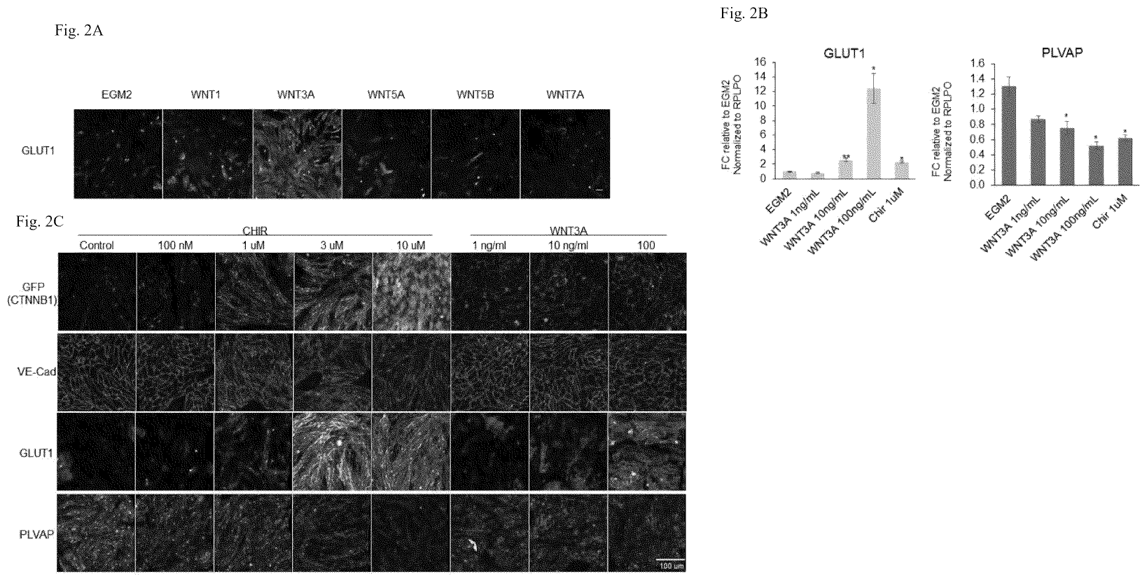

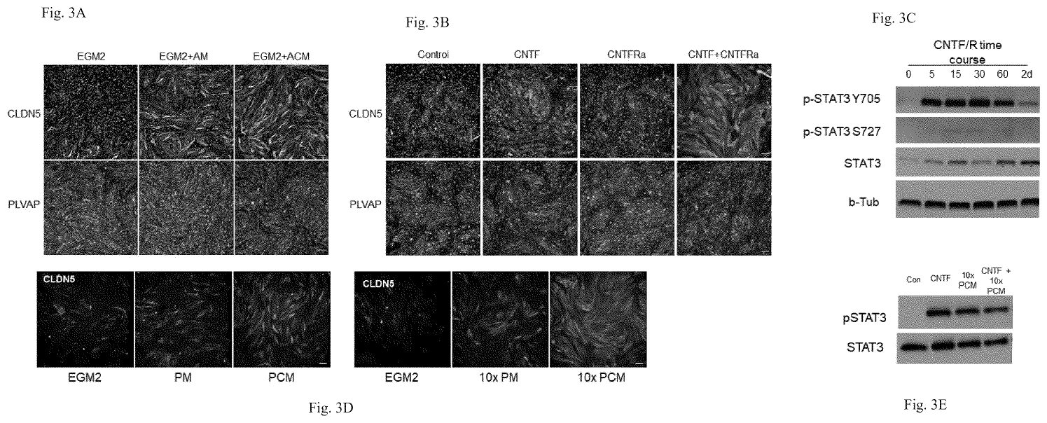

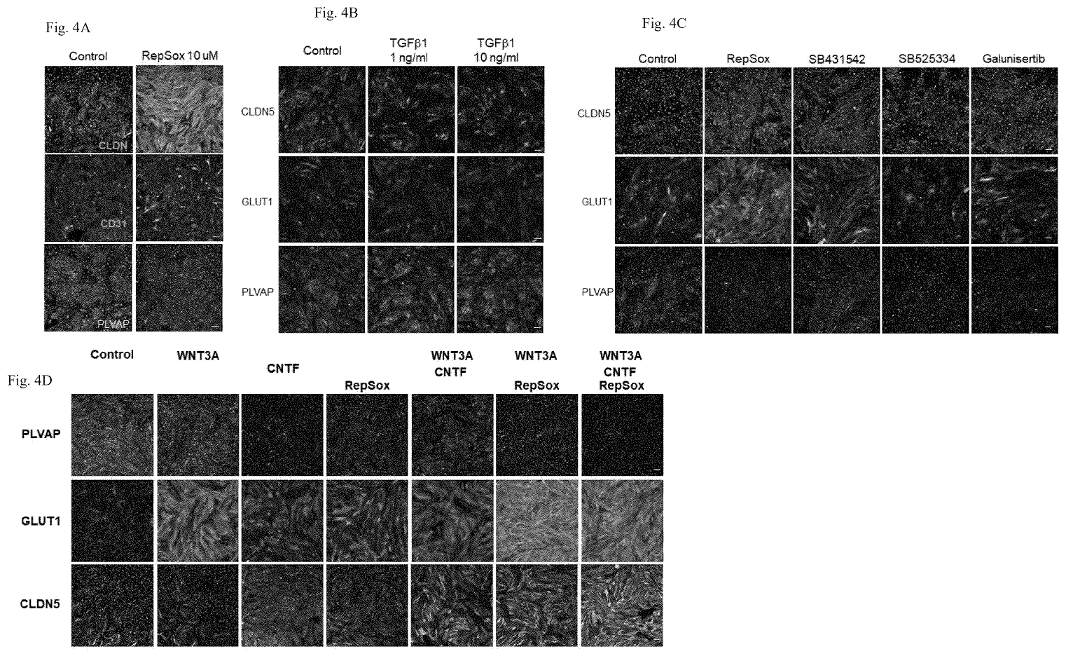

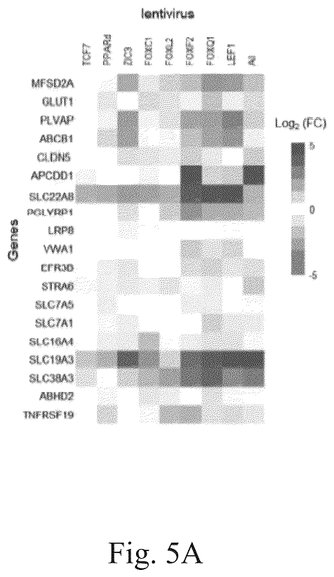

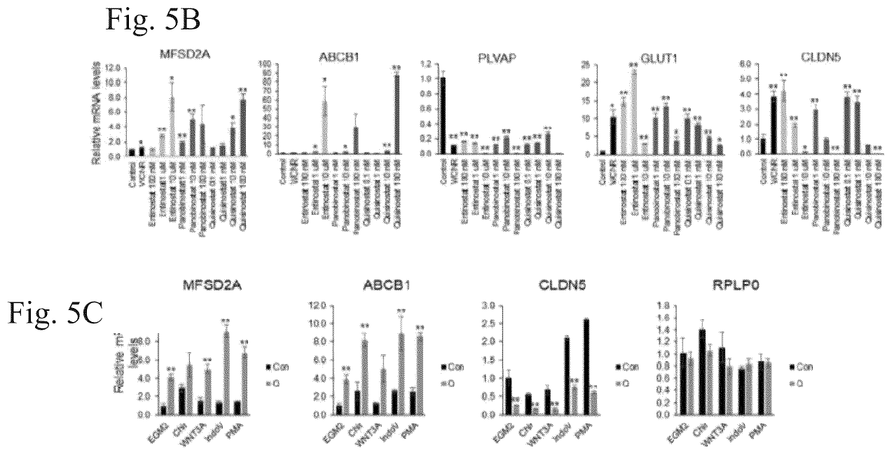

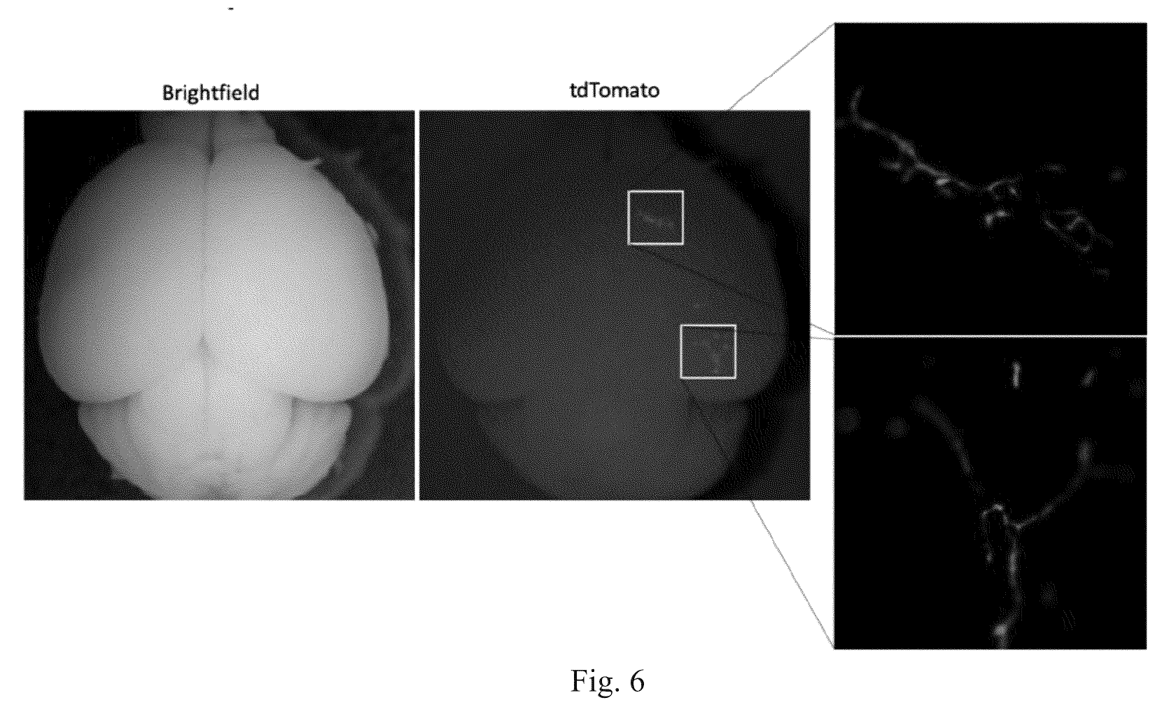

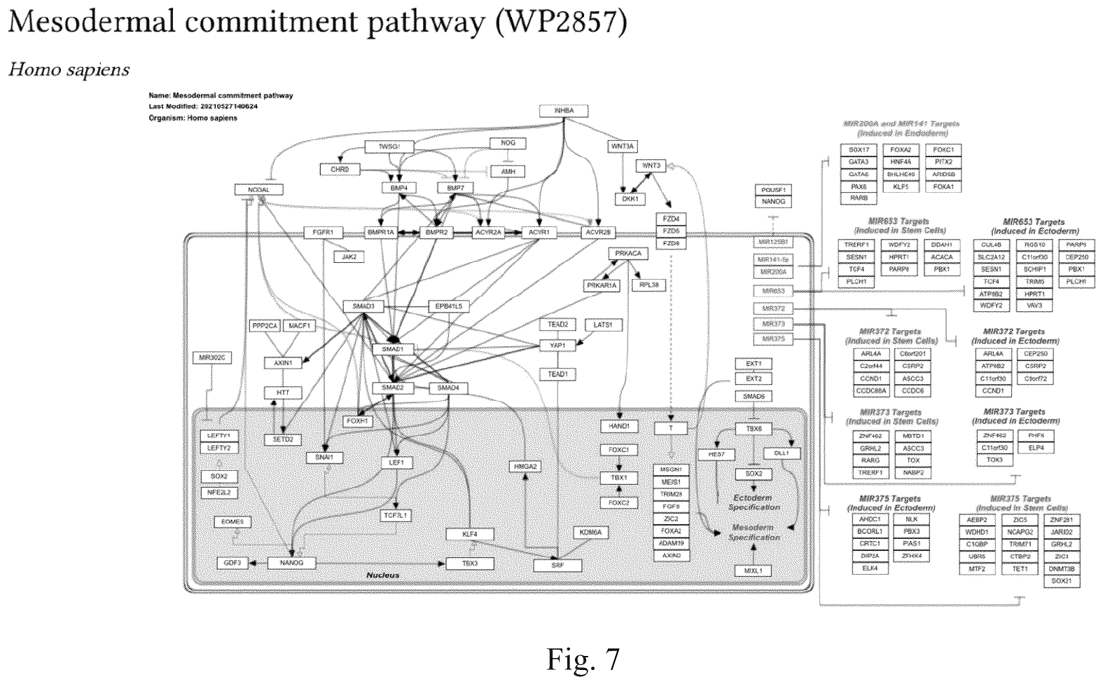

### DETAILED DESCRIPTION OF THE INVENTION

Aspects of the disclosure relate to compositions, methods, kits, and agents for producing non-naturally occurring in vitro-derived peripheral endothelial cells or non-naturally occurring in vitro-derived brain endothelial cells from at least one precursor cell, including pluripotent cells and mesodermally committed cells, and cells produced by those compositions, methods, kits, and agents for use in cell therapies, assays (e.g., drug screening), and various methods of treatment.

### Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a stem cell such as an induced pluripotent stem cell) in a cellular differentiation process.

A “precursor thereof” as the term relates to a non-naturally occurring in vitro-derived brain endothelial cell or peripheral endothelial cell refers to any cell that is capable of differentiating into a non-naturally occurring in vitro-derived brain endothelial cell or peripheral endothelial cell, including for example, a pluripotent stem cell, a definitive mesoderm cell, a progenitor cell, when cultured under conditions suitable for differentiating the precursor cell into the non-naturally occurring in vitro-derived brain endothelial cell or peripheral endothelial cell.

The term “pan EC marker” refers to, without limitation, proteins, peptides, nucleic acids, polymorphism of proteins and nucleic acids, splice variants, fragments of proteins or nucleic acids, elements, and other analytes which are specifically expressed or present in brain or peripheral endothelial cells. Exemplary markers include, but are not limited to, CDH5/VE-CAD, CD31/PECAM1, and SOX17.

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem(ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refer to a pluripotent stem cell artificially derived (e.g., induced or by complete reversal) from a non-pluripotent cell, typically an adult somatic cell, for example, by inducing a forced expression of one or more genes.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as an cardiomyocyte precursor), and then to an end-stage differentiated cell, which plays a characteristic role in a certain tissue type, and may or may not retain the capacity to proliferate further.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cell's differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The term “agent” as used herein means any compound or substance such as, but not limited to, a small molecule, nucleic acid, polypeptide, peptide, drug, ion, etc. An agent can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property or can be selected from a library of diverse compounds.

As used herein, the term “contacting” is intended to include incubating the agent and the cell together in vitro (e.g., adding the agent to cells in culture). In some embodiments, the term “contacting” is not intended to include the in vivo exposure of cells to the compounds as disclosed herein that may occur naturally in a subject (i.e., exposure that may occur as a result of a natural physiological process). The step of contacting can be conducted in any suitable manner. For example, the cells may be treated in adherent culture or in suspension culture. In some embodiments, the cells are treated in conditions that promote cell clustering. Examples of conditions that promote cell clustering include, without limitation, suspension culture in low attachment tissue culture plates, spinner flasks, aggrewell plates.

It is understood that the cells contacted with an agent can also be simultaneously or subsequently contacted with another agent, such as a growth factor or other differentiation agent or environments to stabilize the cells, or to differentiate the cells further. In some embodiments, the cell is contacted with at least two, at least three, at least four, at least five, at least six, at least seven, at least eight, at least nine, or at least 10 agents.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications or replacement of the sugar, as well as (d) internucleoside linkage modifications, including modification or replacement of the phosphodiester linkages. To the extent that such modifications interfere with translation (i.e., results in a reduction of 50% or more in translation relative to the lack of the modification, e.g., in a rabbit reticulocyte in vitro translation assay), the modification is not suitable for the methods and compositions described herein.

In some aspects, the disclosure provides a cell that has been genetically modified to include a detectable marker at a particular locus. It is contemplated that any detectable marker can be inserted into the locus, including for example, a nucleic acid encoding a fluorescent protein (e.g., GFP). Those skilled in the art will appreciate that such genetically modified cells can be used in various screening methods.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.”

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a particular selected population of cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not the particular selected cells as defined by the terms herein.

Similarly, a “substantially pure” or “essentially purified” population of cells refers to a population of cells that contain fewer than about 20%, more preferably fewer than about 15%, 10%, 8%, 7%, most preferably fewer than about 5%, 4%, 3%, 2%, 1%, or less than 1%, of cells that are not the desired cell type as defined by the terms herein.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein to refer to the ability of stem cells to renew themselves by dividing into the same non-specialized cell type over long periods, and/or many months to years. In some instances, proliferation refers to the expansion of cells by the repeated division of single cells into two identical daughter cells.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The term “vector” refers to a carrier DNA molecule into which a DNA sequence can be inserted for introduction into a host cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g EPV and EBV vectors.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

As used herein, the term “transcription factor” refers to a protein that binds to specific parts of DNA using DNA binding domains and is part of the system that controls the transfer (or transcription) of genetic information from DNA to RNA. As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra-or extracellular signals and mechanisms that block or negatively affect cell proliferation.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained for a under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

A “reporter gene” as used herein encompasses any gene that is genetically introduced into a cell that adds to the phenotype of the stem cell. Reporter genes as disclosed in this invention are intended to encompass fluorescent, luminescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny. A reporter gene is generally operatively linked to sequences that regulate its expression in a manner dependent upon one or more conditions which are monitored by measuring expression of the reporter gene. In some cases, expression of the reporter gene may be determined in live cells. Where live cell reporter gene assays are used, reporter gene expression may be monitored at multiple time points, e.g., 2, 3, 4, 5, 6, 8, or 10 or more time points. In some cases, where a live cell reporter assay is used, reporter gene expression is monitored with a frequency of at least about 10 minutes to about 24 hours, e.g., 20 minutes, 1 hour, 2 hours, 3 hours, 4 hours, 5 hours, 6 hours, 7 hours, 8 hours, 9 hours, 10 hours, 12 hours, 18 hours, or another frequency from any integer between about 10 minutes to about 24 hours.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill or injured, or at increased risk of becoming ill relative to an average member of the population and in need of such attention, care, or management.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health.

As used herein, the terms “administering,” “introducing” and “transplanting” are used interchangeably in the context of the placement of cells of the invention into a subject, by a method or route which results in at least partial localization of the introduced cells at a desired site. The cells can be implanted directly to the tissue, or alternatively be administered by any appropriate route which results in delivery to a desired location in the subject where at least a portion of the implanted cells or components of the cells remain viable. The period of viability of the cells after administration to a subject can be as short as a few hours, e.g. twenty-four hours, to a few days, to as long as several years. In some instances, the cells can also be administered in a capsule (e.g., microcapsule) to maintain the implanted cells at the implant location and avoid migration of the implanted cells.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of cells and/or their progeny and/or compound and/or other material other than directly into the central nervous system, such that it enters the animal's system and, thus, is subject to metabolism and other like processes, for example, subcutaneous administration.

The term “tissue” refers to a group or layer of specialized cells which together perform certain special functions. The term “tissue-specific” refers to a source of cells from a specific tissue.

The terms “decrease,” “reduced,” “reduction,” “decrease” or “inhibit” are all used herein generally to mean a decrease by a statistically significant amount. However, for avoidance of doubt, “reduced,” “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

The terms “increased,” “increase” or “enhance” or “activate” are all used herein to generally mean an increase by a statically significant amount; for the avoidance of any doubt, the terms “increased,” “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, for example, in Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM ?-mercaptoethanol. Just before use, human bFGF is added to 4 ng/mL (WO 99/20741, Geron Corp.). Traditionally, ES cells are cultured on a layer of feeder cells, typically fibroblasts derived from embryonic or fetal tissue.

What is claimed is:

1. A method of producing a cell population comprising peripheral endothelial cells in vitro comprising:
(a) inducing one or more pluripotent cells to undergo mesodermal commitment,
(b) contacting the one or more cells that have undergone mesodermal commitment with VEGF, and
(c) purifying and optionally expanding CD144+ cells.

2. The method of claim 1, wherein the one or more pluripotent cells are human cells.

3. The method of claim 1, wherein the one or more pluripotent cells are selected from the group consisting of embryonic stem cells and induced pluripotent stem cells.

4. The method of claim 3, wherein the one or more pluripotent cells are induced pluripotent stem cells.

5. The method of claim 1, wherein the one or more pluripotent cells are cells that express NANOG.

6. The method of any one of claims 1-5, wherein inducing one or more pluripotent cells to undergo mesodermal commitment comprises contacting the one or more pluripotent cells with Chir99021 on day 1.

7. The method of any one of claims 1-5, wherein inducing one or more pluripotent cells to undergo mesodermal commitment comprises contacting the one or more pluripotent cells with BMP4 on day 1.

8. The method of any one of claims 1-5, wherein inducing one or more pluripotent cells to undergo mesodermal commitment comprises contacting the one or more pluripotent cells with Chir99021 and BMP4 on day 1.

9. The method of any one of claims 1-5, wherein mesodermal commitment comprises expression of PAX2 and BRACHYURY.

10. The method of claim 9, wherein PAX2 expression peaks at about day 2.

11. The method of claim 9, wherein BRACHYURY expression peaks at about day 3.

12. The method of any one of claims 1-11, wherein contacting the one or more cells that have undergone mesodermal commitment with VEGF occurs on about day 4.

13. The method of any one of claims 1-12, wherein the cells begin expressing one or more of FLT1/VEGFR1 and KDR/VEGFR2 after being contacted with VEGF.

14. The method of claim 13, wherein expression of FLT1/VEGFR1 and KDR/VEGFR2 peaks on about day 5.

15. The method of any one of claims 1-14, further comprising contacting the one or more cells that have undergone mesodermal commitment with FSK.

16. The method of claim 15, wherein contacting the one or more cells that have undergone mesodermal commitment with FSK occurs on about day 4.

17. The method of any one of claims 1-16, wherein the one or more cells begin to express one or more pan-EC markers on about day 5.

18. The method of claim 17, wherein the one or more pan-EC markers are selected from the group consisting of CDH5/VE-CAD, CD31/PECAM1, and SOX17.

19. The method of any one of claims 1-17, wherein the cells do not significantly express one or more ectodermal epithelial cell markers by day 4.

20. The method of any one of claims 1-17, wherein the cells do not significantly express one or more ectodermal epithelial cell markers by day 5.

21. The method of claim 19 or claim 20, wherein the one or more ectodermal epithelial cell markers is selected from the group consisting of PAX6, MAP2, CDH1 and EPCAM.

22. The method of any one of claims 1-21, wherein purifying CD144+ cells comprises magnetic cell sorting using microbeads.

23. The method of any one of claims 1-22, further comprising purifying CD31+ cells.

24. The method of claim 23, wherein purifying CD31+ cells comprises magnetic cell sorting using microbeads.

25. The method of claim 23 or claim 24, wherein purifying CD144+ and CD31+ cells comprises sequential cell sorting.

26. The method of claim 25, wherein the sequential cell sorting comprises magnetic cell sorting using microbeads.

27. A method of producing a cell population comprising brain endothelial cells in vitro comprising
(a) inducing one or more pluripotent cells to undergo mesodermal commitment,
(b) contacting the one or more cells that have undergone mesodermal commitment with VEGF,
(c) purifying and optionally expanding CD144+ cells, and
(d) modulating one or more signaling pathways selected from the group consisting of the Wnt signaling pathway, the TGF-beta signaling pathway, and the STAT3 signaling pathway in the purified and optionally expanded CD144+ cells.

28. The method of claim 27, wherein the one or more pluripotent cells are human cells.

29. The method of claim 27, wherein the one or more pluripotent cells are selected from the group consisting of embryonic stem cells and induced pluripotent stem cells.

30. The method of claim 29, wherein the one or more pluripotent cells are induced pluripotent stem cells.

31. The method of claim 27, wherein the one or more pluripotent cells are cells that express NANOG.

32. The method of any one of claims 27-31, wherein inducing one or more pluripotent cells to undergo mesodermal commitment comprises contacting the one or more pluripotent cells with Chir99021 on day 1.

33. The method of any one of claims 27-31, wherein inducing one or more pluripotent cells to undergo mesodermal commitment comprises contacting the one or more pluripotent cells with BMP4 on day 1.

34. The method of any one of claims 27-31, wherein inducing one or more pluripotent cells to undergo mesodermal commitment comprises contacting the one or more pluripotent cells with Chir99021 and BMP4 on day 1.

35. The method of any one of claims 27-31, wherein mesodermal commitment comprises expression of PAX2 and BRACHYURY.

36. The method of claim 35, wherein PAX2 expression peaks at about day 2.

37. The method of claim 35, wherein BRACHYURY expression peaks at about day 3.

38. The method of any one of claims 27-37, wherein contacting the one or more cells that have undergone mesodermal commitment with VEGF occurs on about day 4.

39. The method of any one of claims 27-38, wherein the cells begin expressing one or more of FLT1/VEGFR1 and KDR/VEGFR2 after being contacted with VEGF.

40. The method of claim 39, wherein expression of FLT1/VEGFR1 and KDR/VEGFR2 peaks on about day 5.

41. The method of any one of claims 27-40, further comprising contacting the one or more cells that have undergone mesodermal commitment with FSK.

42. The method of claim 41, wherein contacting the one or more cells that have undergone mesodermal commitment with FSK occurs on about day 4.

43. The method of any one of claims 27-42, wherein the one or more cells begin to express one or more pan-EC markers on about day 5.

44. The method of claim 43, wherein the one or more pan-EC markers are selected from the group consisting of CDH5/VE-CAD, CD31/PECAM1, and SOX17.

45. The method of any one of claims 27-43, wherein the cells do not significantly express one or more ectodermal epithelial cell markers by day 4.

46. The method of any one of claims 27-43, wherein the cells do not significantly express one or more ectodermal epithelial cell markers by day 5.

47. The method of claim 45 or claim 46, wherein the one or more ectodermal epithelial cell markers is selected from the group consisting of PAX6, MAP2, CDH1 and EPCAM.

48. The method of any one of claims 27-47, wherein purifying CD144+ cells comprises magnetic cell sorting using microbeads.

49. The method of any one of claims 27-48, further comprising purifying CD31+ cells.

50. The method of claim 49, wherein purifying CD31+ cells comprises magnetic cell sorting using microbeads.

51. The method of claim 49 or claim 50, wherein purifying CD144+ and CD31+ cells comprises sequential cell sorting.

52. The method of claim 51, wherein the sequential cell sorting comprises magnetic cell sorting using microbeads.

53. The method of any one of claims 27-52, wherein the one or more signaling pathways is the Wnt signaling pathway

54. The method of any one of claims 27-52, wherein the one or more signaling pathways is the TGF-beta signaling pathway.

55. The method of any one of claims 27-52, wherein the one or more signaling pathways is the STAT3 signaling pathway.

56. The method of claim 53, wherein modulation of the Wnt signaling pathway is achieved by contacting the purified and optionally expanded CD144+ cells with one or more agents selected from the group consisting of EGM2, WNT1, WNT3A, WNT5A, WNT5B and WNT7A.

57. The method of claim 56, wherein modulation of the Wnt signaling pathway upregulates GLUT1.

58. The method of claim 56, wherein modulation of the Wnt signaling pathway downregulates PLVAP.

59. The method of any one of claims 27-52, wherein modulation of the one or more signaling pathways is achieved by contacting the purified and optionally expanded CD144+ cells with one or more agents selected from the group consisting of EC medium, ciliary neurotrophic factor (CNTF), CNTF receptor alpha (CNTFRa), pericyte medium (PM), pericyte conditioned medium (PCM), concentrated PM, and concentrated PCM.

60. The method of claim 59, wherein modulation increases CLDN5 expression.

61. The method of claim 54, wherein modulation of the TGF beta signaling pathway is achieved by contacting the purified and optionally expanded CD144+ cells with one or more TGFBR1 inhibitors.

62. The method of claim 54, wherein modulation of the TGF beta signaling pathway is achieved by contacting the purified and optionally expanded CD144+ cells with one or more agents selected from the group consisting of RepSox, SB431542, SB525334 and Galunisertib. 62 The method of claim 61, wherein modulation increases CLDN5 expression.

63. The method of any one of claims 27-52, wherein two of the Wnt signaling pathway, the TGF beta signaling pathway, and the STAT3 signaling pathway are modulated.

64. The method of any one of claims 27-52, wherein the Wnt signaling pathway, the TGF beta signaling pathway, and the STAT3 signaling pathway are all modulated.

65. The method of any one of claims 27-64, further comprising inhibiting HDAC expression in the purified and optionally expanded CD144+ cells.

66. The method of claim 65, wherein inhibiting HDAC expression comprises contacting the purified and optionally expanded CD144+ cells with one or more agents selected from the group consisting of entinostat, panobinostat, and quisinostat.

67. The method of any one of claims 27-66, further comprising increasing expression of one or more brain EC-specific transcription factors in the purified and optionally expanded CD144+ cells.

68. The method of claim 67, wherein the one or more EC-specific transcription factors is selected from the group consisting of TCF7, PPARd, ZIC3, FOXC1, FOXL2, FOXF2, FOXQ1, and LEF1.

69. A non-naturally occurring in vitro-derived peripheral endothelial cell produced by any one of the methods of claims 1-26.

70. A non-naturally occurring in vitro-derived brain endothelial cell produced by any one of the methods of claims 27-68.

71. The non-naturally occurring in vitro-derived brain endothelial cell of claim 70 characterized by one or more of EC marker expression, monolayer formation, increased expression of CLDN5 in comparison with a non-EC cell, increased expression of OCLN in comparison with a non-EC cell, increased expression of GLUT1 in comparison with a non-EC cell, increased expression of ABCB1 in comparison with a non-EC cell, increased expression of MFSD2A in comparison with a non-EC cell, and decreased expression of PLVAP in comparison with a non-EC cell.

72. A non-naturally occurring in vitro-derived peripheral endothelial cell.

73. A non-naturally occurring in vitro-derived brain endothelial cell.

74. The non-naturally occurring in vitro-derived brain endothelial cell of claim 73 characterized by one or more of EC marker expression, monolayer formation, increased expression of CLDN5 in comparison with a non-EC cell, increased expression of OCLN in comparison with a non-EC cell, increased expression of GLUT1 in comparison with a non-EC cell, increased expression of ABCB1 in comparison with a non-EC cell, increased expression of MFSD2A in comparison with a non-EC cell, and decreased expression of PLVAP in comparison with a non-EC cell.
